(19) United States
(12) Patent Application Publication
(10) Pub. No.: US 2012/0101307 A1
(43) Pub. Date: Apr. 26, 2012

(54) PROCESS FOR THE PREPARATION OF 1-METHYL-1-ALKOXYCYCLOPENTANES

(75) Inventors: Andreas LANVER, Mannheim (DE); Klaus Ebel, Lampertheim (DE); Rainer Klopsch, Worms (DE); Werner Bertleff, Viernheim (DE); Richard Dehn, Ludwigshafen (DE); Joaquim Henrique Teles, Waldsee (DE); Helmut Kronemayer, Heidelberg (DE); Marcus Georg Schrems, Ludwigshafen (DE)

(73) Assignee: BASF SE, Ludwigshafen (DE)

(21) Appl. No.: 13/279,915

(22) Filed: Oct. 24, 2011

Related U.S. Application Data
(60) Provisional application No. 61/406,181, filed on Oct. 25, 2010.

(51) Int. Cl. C07C 41/01

(52) U.S. Cl. 568/579

(57) ABSTRACT

Process for the preparation of cyclopentane derivatives of the formula I

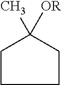

in which R is a C1- to C10-alkyl group, which comprises reacting cyclohexanol or cyclohexene or mixtures of both compounds to give 1-methyl-1-cyclopentene (1st stage) and subsequently adding a C1- to C10-alkanol onto the double bond of the 1-methyl-1-cyclopentene (2nd stage).

The present application includes by reference the provisional U.S. application 61/406,181, filed Oct. 25, 2010.

The invention relates to a process for the preparation of cyclopentane derivatives of the formula I

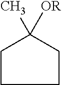

in which R is a C₁-C₁₀-alkyl group, which comprises reacting cyclohexanol or cyclohexene or mixtures of both compounds to 1-methyl-1-cyclopentene (1st stage) and subsequently adding a C₁- to C₁₀-alkanol, onto the double bond of the 1-methyl-1-cyclopentene (2nd stage).

Cyclopentane derivatives such as 1-methyl-1-hydroxycyclopentane are of importance as starting materials for chemical syntheses of a very wide variety of compounds.

It is known from U.S. Pat. No. 5,498,802 to prepare 1-methyl-1-hydroxycyclopentane in three separate reaction steps, with cyclohexanol being dehydrated to give cyclohexene in the first step, cyclohexene being isomerized to give 1-methylcyclopentene in the 2nd step, and water being added on to 1-methylcyclopentene in the presence of isopropanol as solvent in the third step.

According to example 1 of U.S. Pat. No. 5,498,802, cyclohexanol dissolved in methanol is dehydrated at 250° C. over silicon dioxide to give cyclohexene. In the event of complete cyclohexanol conversion, a cyclohexene yield in the first step of 97.5% was achieved.

In the second step, cyclohexene is isomerized at 400° C. in the gas phase over silicon dioxide to give 1-methylcyclopentene. The 1-methylcyclopentene yield was 60.3% (example 2).

In the third step, a mixture of pure 1-methylcyclopentene, isopropanol and water is passed at 80° C. over Amberlyst15 resin. The 1-methylcyclopentene conversion was 41.9%, the yield of 1-methyl-1-hydroxycyclopentane 22.9% (example 3).

A disadvantage of the process according to U.S. Pat. No. 5,498,802 is the number of reaction steps and the low overall yield of 1-methyl-1-hydroxycyclopentane of only 13%, calculated over all part-steps. For the catalytic addition reaction of water onto 1-methylcyclopentene to give 1-methyl-1-hydroxycyclopentane pure 1-methylcyclopentene is used.

In the case of the preparation of 1-methyl-1-hydroxycyclopentane, it is also fundamentally disadvantageous that during the reaction a two phase system of 1-methylcyclopentene and water, which is added onto the double bond, is present and therefore in general an additional polar organic solvent is required as solubilizer. This makes both the implementation of the reaction and also the work-up more difficult. In this connection, it is also disadvantageous that the need for a solubility promoter and the formation of azeotropes of water and solubility promoters prevent reactive distillation processes being able to be used effectively.

Neftekhimiya (1991), 31 (3), pages 386 to 390 (No. 12) describes the conversion of cyclohexanol to 1-methylcyclopentene in one step. Cyclohexanol is converted to methylcyclopentenene at 450° C. in the gas phase over chlorine-doped aluminum oxide.

U.S. Pat. No. 4,661,639 relates to a process for the preparation of cyclic alcohols by addition of water onto cyclic olefins using modified aluminum silicate catalysts.

1-Methyl-1-hydroxycyclopentane is a reactive starting material for further syntheses. The disadvantages described above for the preparation of 1-methyl-1-hydroxycyclopentane lead to a requirement for an intermediate which, as far as possible, has the same reactivity as 1-methyl-1-hydroxycyclopentane and can be used accordingly, but can be prepared by a simpler process.

Accordingly, the process defined above has been found.

In this application, the following seven compounds are referred to as follows:

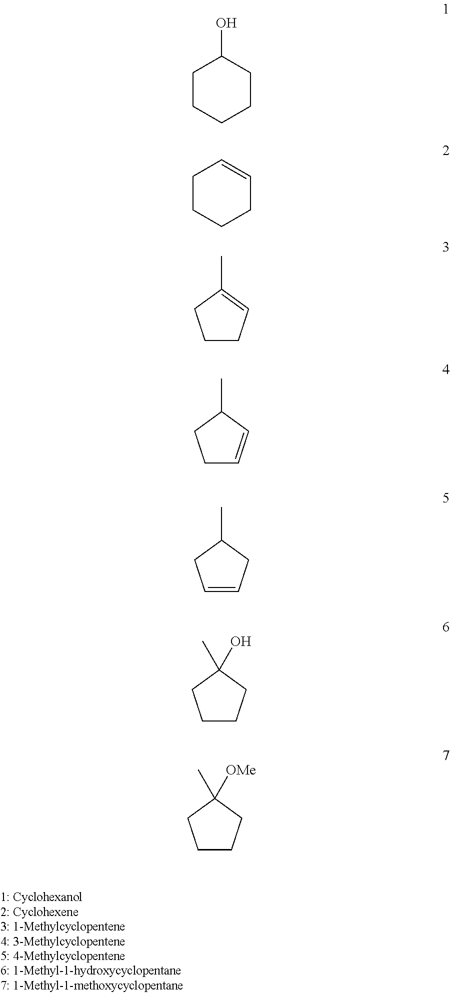

### The 1St Stage (Preparation of 1-Methylcyclopentene)

The starting compound of the reaction in the 1st stage (preparation of 1-methylcyclopentene) is cyclohexanol or cyclohexene or a mixture of the two.

The reaction is a gas-phase reaction.

Starting from cyclohexene, a rearrangement to give 1-methylcyclopentene takes place; starting from cyclohexanol, an elimination of water to give cyclohexene takes place in the 1st stage and then the subsequent rearrangement to 1-methylcyclopentene according to the following schematically represented reaction sequence:

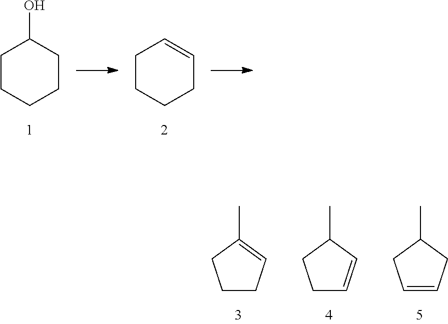

This gas-phase reaction is known per se.

Cyclohexene as starting compound can also be obtained by means of a separate dehydration of cyclohexanol carried out beforehand or any other desired process, e.g. by partial hydrogenation of benzene by the Asahi process.

A preferred starting compound is cyclohexanol or a mixture of cyclohexanol and cyclohexene, where the molar ratio of cyclohexanol to cyclohexene is 1:0.1 to 0.1:1.

The conversion of cyclohexanol to cyclohexene is generally complete.

The conversion can take place discontinuously or preferably continuously, i.e. with the continuous introduction of the starting materials and continuous discharge of the products.

In the process according to the invention, a recycling of the by-products 3-methylcyclopentene and 4-methylcyclopentene and, optionally, also unreacted starting compounds takes place. The term “starting compounds” therefore comprises hereinbelow always also recycled compounds, e.g. recycled by-products and recycled, unreacted starting compounds, as is explained in more detail below.

The conversion preferably takes place in the gas phase in the presence of acidic catalysts. The gas-phase reaction can be carried out in reactors such as stirred reactors or tubular reactors.

The acidic catalysts can be arranged in the reactor as fixed bed or fluidized bed. A required inertization of the catalysts can be carried out with a carrier gas, such as e.g. nitrogen or argon.

Suitable solid acidic catalysts are e.g. SiO2, Al2O3, mixtures of SiO2 and Al2O3, alumosilicates, ZrO2, TiO2 or zeolites.

Suitable catalysts for the conversion of cyclohexanol or cyclohexene to 1-methylcyclopentene are also naturally occurring or synthetically produced zeolites.

The catalyst velocity is preferably 0.05 to 3, preferably 0.1 to 2, particularly preferably 0.2 to 1 kg of starting compounds per liter of catalyst and hour.

The residence time is in particular from 1 to 50 seconds, preferably 5 to 15 seconds.

The conversion can take place at temperatures of from 250 to 500, preferably 300 to 450, particularly preferably from 400 to 450° C.

The reaction pressure is not critical. It can be for example 0.1 to 10 bar, preferably 1 to 5 bar.

The product mixture obtained during the conversion comprises, besides the desired product 1-methylcyclopentene, also the following compounds:
the by-products 3-methylcyclopentene and 4-methylcyclopentene (double-bond isomers of 1-methylcyclopentene),
unreacted cyclohexene and
optionally water (if cyclohexanol has been used as starting material).

In general, during the conversion, 1 to 50 parts by weight, in particular 2 to 40 parts by weight, often 5 to 30 or 10 to 30 parts by weight, of 3-methylcyclopentene are formed per 100 parts by weight of 1-methylcyclopentene obtained.

In general, during the reaction, 1 to 50 parts by weight, in particular 1 to 30 parts by weight, often 1 to 20 or 2 to 20 parts by weight, of 4-methylcyclopentene are formed per 100 parts by weight of 1-methylcyclopentene obtained.

The gaseous reaction discharge is condensed. This can take place e.g. by adding an organic solvent such as toluene (quenching). When co-using cyclohexanol as starting material, the condensate consists of two liquid phases, one aqueous and one organic phase. The two phases are separated. The organic phase comprises the aforementioned product mixture, in particular the product of value 1-methylcyclopentene. The water phase is removed from the system.

The separation of the two liquid phases can take place by gravimetric phase separation. Suitable phase separation vessels are, for example, customary standard apparatuses and standard methods. Alternatively, water can be separated off from the condensed reaction discharge also by means of an azeotropic distillation.

The organic phase, which generally comprises unreacted cyclohexene (boiling point 83° C.), 1-methylcyclopentene (boiling point 76° C.), and also the two isomers 3-methylcyclopentene and 4-methylcyclopentene (boiling points 65-66° C.), can be worked up by distillation. The above boiling points are applicable for standard pressure; 1-methylcyclopentene can then be separated off from this organic phase and be used as end product in the desired manner. This leaves an organic residue, which comprises unreacted cyclohexene and the two isomers 3-methylcyclopentene and 4-methylcyclopentene.

The 2Nd Stage

In formula I, R is preferably a C1- to C4-alkyl group e.g. a methyl, ethyl, n-propyl or n-butyl group; R is particularly preferably a methyl group.

During the subsequent reaction in the 2nd stage, the addition of the alkanol, which corresponds to the desired radical R, onto the double bond of the 1-methylcyclopentene takes place.

In accordance with the desired radical X in formula I, the alkanol is a C1- to C10-alkanol, in particular a C1- to C4-alkanol, e.g. methanol, ethanol, n-propanol or n-butanol. It is particularly preferably methanol.

In the 2nd stage, 1-methylcyclopentene is preferably reacted in the liquid phase in the presence of acidic catalysts with the desired alkanol to give cyclopentane derivatives of the formula I, particularly preferably with methanol to give 1-methyl-1-methoxycyclopentane.

The co-use of additional solvents is not necessary.

The reaction can be carried out at temperatures of for example 20 to 100° C., preferably 50 to 90° C., particularly preferably 40 to 90° C.

The reaction pressure is not critical. It can be for example 0.1 to 10 bar, preferably 1 to 5 bar.

The molar ratio of 1-methylcyclopentene to the alkanol can be e.g. 1:10 to 10:1. In one preferred embodiment, the alkanol is used in a molar excess; the molar ratio of 1-methylcyclopentene to the alkanol is then in particular 1:1 to 1:10, particularly preferably 1:2 to 1:5.

The acidic catalysts used are preferably solid acidic catalysts, such as e.g. highly acidic ion exchangers or zeolites, as are also specified in U.S. Pat. No. 5,498,802.

The solid acidic catalysts can either be arranged in a fixed manner in a reactor or are suspended in the liquid phase.

The catalyst velocity can be for example 20 to 1, preferably 15 to 3, particularly preferably 5 to 3 kg, of methylcyclopentenes per liter of catalyst and hour.

The residence time can be for example 5 minutes to 2 hours, in particular 5 minutes to 2 hours and preferably 10 minutes to 30 minutes.

The reaction discharge from stage 2 comprises the desired cyclopentane derivatives of the formula I, unreacted 1-methylcyclopentene, optionally isomers thereof as by-products and, optionally, unreacted alkanol. If the acidic catalyst was suspended in the reaction mixture, it can be separated off by filtration and returned to synthesis step 2.

The reaction discharge can be worked up by distillation.

For the purification and separation off of the desired cyclopentane derivative of the formula I, fractional distillations can be carried out.

Preferably, the reaction in the 2nd stage (addition reaction of the alkanol) and a distillative separation of the product mixture take place in a single process step by means of a reactive distillation.

The product or product mixture is produced here as bottom product on account of the boiling point difference between the resulting cyclopentane derivative of the formula I (preferably 1-methyl-1-methoxycyclopentane) and the starting materials. By means of a reactive distillation, high conversions are possible for equilibrium reactions since the product which forms is removed directly.

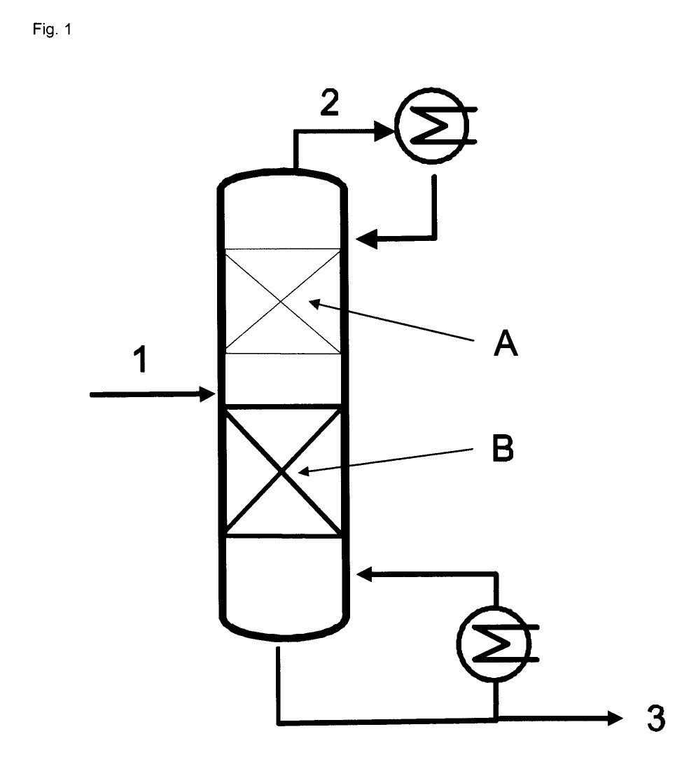

### FIG. 1

In a distillation apparatus suitable for continuous operation, as in FIG. 1, the starting materials (here C1 to 010 alkanol, preferably methanol, and 1-methylcyclopentene, and, if appropriate, recycled by-products) can be introduced into the apparatus in gaseous or liquid form (stream 1 in FIG. 1). In the column, the conversion to the cyclopentane derivative of the formula I (preferably 1-methyl-1-methoxycyclopentane) takes place. The readily volatile starting compounds are condensed at the top of the column and returned to the column (stream 2). The less volatile cyclopentane derivative of the formula I (preferably 1-methyl-1-methoxycyclopentane) and less volatile by-products accumulate in the bottom of the column and can be drawn off (stream 3).

The distillation apparatus can preferably comprise customary internals for promoting distillative separation (packings or column trays) and the acidic catalyst, e.g. in the form of a fixed bed or fluidized bed (see internals A and B in FIG. 1).

The process in the 2nd stage can be carried out discontinuously or continuously; preferably, the process in the 2nd stage is carried out continuously.

Preferably, the overall process comprising the 1st and 2nd stage is therefore a process that is carried out continuously.

### The Recycle

An essential feature of the two-stage process according to the invention for the preparation of cyclopentane derivatives is that 3-methylcyclopentene and 4-methylcyclopentene, and, optionally, unreacted starting compounds in the product mixture from the 1st or 2nd stage are returned to the reaction if the 1st stage.

Recycle from 1St Stage

In one embodiment, 3-methylcyclopentene and 4-methylcyclopentene and, optionally, unreacted starting materials, i.e. cyclohexene, from the product mixture in the 1st stage are returned to the reaction in the 1st stage.

In this embodiment, therefore, 1-methylcyclopentene is separated off from the product mixture in the 1st stage as described above and passed to the 2nd stage, while 3-methylcyclopentene and 4-methylcyclopentene and preferably also the unreacted cyclohexene are returned to the reaction in the 1st stage. In particular, the entire organic residue after separating off the 1-methylcyclopentene is returned.

If cyclohexanol is used as starting compound alone or in a mixture with cyclohexene, the product mixture comprises water from the dehydration of cyclohexanol to cyclohexene. Water is produced as a separate aqueous phase and can easily be separated off from the organic phase, as is described above. The aqueous phase is discarded and not returned to the reaction in the 1st stage.

In this embodiment, in addition to the recycle in the 1st stage, a recycle can also take place in the 2nd stage. In particular, unreacted starting materials from the 2nd stage, i.e. 1-methylcyclopentene and unconsumed alkanol, are returned to the reaction in the 2nd stage.

Recycle from Product Mixture of the 2Nd Stage

In a further embodiment of the two-stage process, after the 1st stage, no separation off and recycle of 3-methylcyclopentene and 4-methylcyclopentene takes place. Instead, 3-methylcyclopentene and 4-methylcyclopentene from the product mixture in the 2nd stage is returned to the reaction in the 1st stage.

In this embodiment, the product mixture from the 1st stage is condensed and the organic phase, without separating off the by-products 3-methylcyclopentene and 4-methylcyclopentene and without separating off the unreacted starting compounds (cyclohexene), is fed to the further reaction in the 2nd stage. If an aqueous phase is produced during the condensation of the product mixture on account of the (co-)use of cyclohexanol, this is preferably separated off and discarded.

The product mixture obtained in the 2nd stage accordingly comprises
the desired cyclopentane derivative of the formula I,
unreacted 1-methylcyclopentene and alkanol (starting materials for stage 2)
the by-products from stage 1: 3-methylcyclopentene and 4-methylcyclopentene
unreacted starting materials from stage 1: cyclohexene

From the product mixture of the 2nd stage, the desired 1-methyl-1-alkoxycyclopentane derivative can be separated off from the aforementioned compounds by fractional distillation as high-boiling components and, if necessary, be purified again by distillation.

Unreacted alkanol is preferably likewise separated off.

This then leaves as residue:
unreacted 1-methylcyclopentene (starting materials for stage 2)
the by-products from stage 1: 3-methylcyclopentene and 4-methylcyclopentene
unreacted starting materials from stage 1: cyclohexene

In this embodiment, these compounds are then returned to the reaction in the 1st stage; preferably the entire residue mentioned above is returned to the 1st stage.

The two-stage process according to the invention for the preparation of cyclopentane derivatives of the formula I (1-methyl-1-alkoxycyclopentanes) is suitable in particular for a continuous process procedure. The yields of 1-methylcyclopentene (product of the 1st stage) or of the desired 1-methyl-1-alkoxycyclopentane (product of the 2nd stage) are high.

The process can be carried out easily; in contrast to the preparation of 1-methyl-1-hydroxycyclopentane, an additional solvent is not required and reactive distillation processes can be used effectively.

By means of the process according to the invention it is possible, in a simple and effective manner, to obtain compounds which can be used as alternative to the reactive 1-methyl-1-hydroxycyclopentane for organic syntheses.

The recycling of 3-methylcyclopentene, 4-methylcyclopentene and cyclohexene does not lead to an increased formation of by-products.

In particular, it can be established that the isomers 3-methylcyclopentene and 4-methylcyclopentene rearrange to the desired compound 1-methylcyclopentene upon recycling to the 1st stage and increase the yield.

Furthermore, it can be established that a presence of the isomers 3-methylcyclopentene and 4-methylcyclopentene in the 2nd stage is not disadvantageous and no or barely any new by-products as a result of an addition of alkanol onto these isomers or else onto cyclohexene are observed.

The following working examples relating to stage 1 (Examples 1 to 3) show that the reactions take place with high selectivity. Example 5 shows the conversion, that takes place upon recycling, of the undesired by-products 3-methylcyclopentene and 4-methylcyclopentene to give the desired 1-methylcyclopentene.

Examples 6 to 9 are comparative examples and describe the preparation of 1-methyl-1-hydroxycyclopentane with co-use of isopropanol as solvent.

Examples 10 to 13 according to the invention exhibit the advantageous preparation of 1-methyl-1-methoxycyclopentane. Example 14 shows that the presence of 3-methylcyclopentene, 4-methylcyclopentene and cyclohexene in the 2nd stage does not lead to new by-products.

### EXAMPLES

The compounds below are referred to in the examples by the chemical name stated below or simply by the associated number.

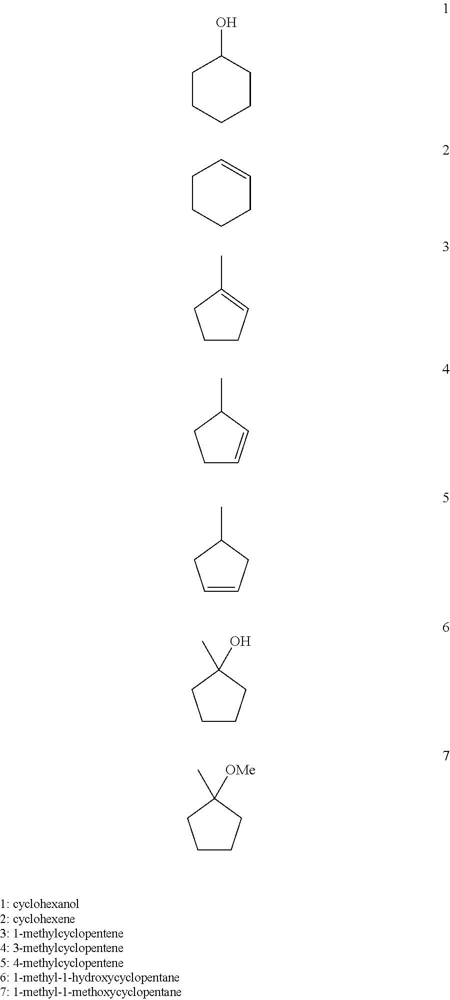

### Abbreviations Used: GC Area %: Gas Chromatography Area Percent

### A) Gas-Phase Reaction of Cyclohexanol to 1-Methylcyclopentene

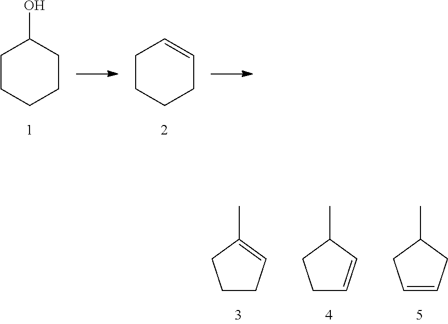

### Examples 1-3

A glass reactor (internal diameter 27 mm; height 500 mm) with electrical heating and temperature measurement was charged with a catalyst amount of ca. 300 ml. The starting material (1) was continuously introduced in trickle mode. In the bottom of the glass reactor, condensation was carried out in a 1 l flask with attached condenser using toluene as quenching liquid. The carrier gas used was nitrogen. The aqueous phase was separated off from the two-phase mixture in the separating funnel.

The organic phase was analyzed by GC.

| | | | | | Composition in GC | | | | |
|---|---|---|---|---|---|---|---|---|---|
| | | T | Carrier | Space | area % [a] | | | | |
| Example | Catalyst | [° C.] | gas N2 | velocity | 4 | 5 | 3 | 2 | 1 |
| 1 | SiO₂ | 400 | 5 l/h | 371 g/kg/h | 11 | 4 | 46 | 32 | 0 |
| 2 | SiO₂ + | 400 | 3 l/h | 112 g/kg/h | 10 | 4 | 54 | 28 | 0 |
| | 20% | | | | | | | | |
| | H₃PO₄ | | | | | | | | |
| 3 | SiO₂ + | 450 | 3 l/h | 117 g/kg/h | 11 | 4 | 58 | 20 | 0 |
| | 20% | | | | | | | | |
| | H₃PO₄ | | | | | | | | |

[a] Toluene deducted

### Example 4

In a column of height 120 cm containing 3×3 mm mesh rings (ca. 60 theoretical plates), a mixture consisting of the compounds 2, 3, 4 and 5 was subjected to discontinuous fractional distillation at atmospheric pressure. The top temperature was a constant 72° C. as compound 3 passed over.

| | | Compounds [in GC area %] | | | | | Boiling point |
|---|---|---|---|---|---|---|---|
| Sample | g | 4 | 5 | 3 | 2 | toluene | [° C.] |
| Feed | 724 | 8 | 3 | 33 | 40 | 13 | |
| Fr.1 | 50 | 68 | 25 | 3 | 0 | 0 | 63-66° C. |
| Fr.2 | 24 | 54 | 24 | 11 | 0 | 0 | |
| Fr.3 | 15 | 22 | 12 | 52 | 0 | 0 | |
| Fr.4 | 14 | 10 | 6 | 75 | 0 | 0 | |
| Fr.5 | 25 | 4 | 2 | 88 | 0 | 0 | 72° C. |
| Fr.6 | 21 | 1 | 1 | 96 | 0 | 0 | |
| Fr.7 | 21 | 0 | 0 | 98 | 0 | 0 | |
| Fr.8 | 37 | 0 | 0 | 99 | 0 | 0 | |
| Fr.9 | 34 | 0 | 0 | 99 | 1 | 0 | |
| Fr.10 | 30 | 0 | 0 | 97 | 2 | 0 | |
| Bottom | 393 | 0 | 0 | 2 | 68 | 27 | 83° C. |

This example shows the ability of compound 3 to be separated off by distillation from the resulting product mixture in the 1st stage.

### Example 5

The same experimental set-up as in Examples 1-3 was used (catalyst: SiO₂+20% H₃PO₄). The feed substance used was the following mixture: compounds 3 (11 GC area %), 4 (56 GC area %), 5 (24 GC area %). At a temperature of 400° C., a carrier gas stream (N2) of 3 l/h and a catalyst space velocity of 146 g/kg/h, following condensation in toluene, the following mixture was obtained: compounds 3 (63 GC area %), 4 (12 GC area %), 5 (5 GC area %).

The experiment demonstrates that compounds 4 and 5 can be isomerized again into the desired product 3. Upon recycling these isomers, the yield is therefore increased accordingly.

### B) Hydration of 1-Methylcyclopentene—for Comparison

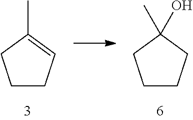

### Examples 6-9

### Comparative Examples

In a continuously operated glass reactor (internal diameter 25 mm; height 200 mm), an ion exchanger (Amberlyst type) was introduced as catalyst. In trickle mode, at a constant internal volume, the mixture consisting of 1-methylcyclopentene, isopropanol (iPrOH) and water was introduced from a storage container via a membrane pump.

The product mixture was analyzed by GC. The yields and conversions were calculated from GC % by weight results.

| | Feed | | | | | | | | |
|---|---|---|---|---|---|---|---|---|---|
| | (3) | iPrOH | H₂O | | | Residence | Conversion | Yield | Selectivity |
| | [% by | [% by | [% by | | T | time | (3) | (6) | (6) |
| Ex. | wt.] | wt.] | wt.] | Catalyst | [° C.] | [min] | [%] | [%] | [%] |
| 6 | 8 | 53 | 39 | Amberlyst 36 | 70 | 22 | 57 | 24 | 42 |
| 7 | 8 | 53 | 39 | Amberlyst 15 | 55 | 36 | 61 | 30 | 49 |
| 8 | 4 | 55 | 41 | Amberlyst 15 | 55 | 11 | 43 | 28 | 65 |
| 9 | 4 | 55 | 41 | Amberlyst 15 | 55 | 48 | 59 | 39 | 66 |

C) Etherification of 1-Methylcyclopentene with Methanol

### Example 10

### Preparation of 1-methoxy-1-methylcyclopentane

### Molar Ratio Olefin:Methanol=1/1.9

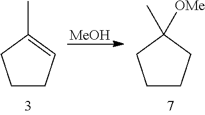

1-Methylcyclopentene (99.2 g, 1.21 mol) and methanol (75.0 g, 2.34 mol) were introduced as initial charge in a 3-neck flask under nitrogen. A Soxhlet attachment with an extraction thimble filled with 15.0 g of Amberlyst 15 was placed on the flask and the mixture was heated to reflux (oil bath: 85° C.). After a total of 67 h, the oil bath was removed and the reaction mixture was cooled. A GC sample was taken, which revealed a conversion of ca. 94%.

### Composition of the Product Mixture:

MeOH: 19.7%

3 (1-methylcyclopentene): 4.6%

7 (ether): 72.5%

By using a Soxhlet, the following effect is achieved if the temperature is adjusted to 85° C.: the two starting materials methylcyclopentene and methanol boil and then condense at the reflux condenser. The condensed mixture then passes into the reaction zone with the ion exchanger and reacts to give the product 1-methoxy-1-methylcyclopentane. Since its boiling temperature is above 85° C., the product can be enriched in the bottom to virtually complete conversion.

### Example 11

### Preparation of 1-methoxy-1-methylcyclopentane

### Olefin:MeOH=1/5.2

Procedure analogous to Example 10, using

99.0 g (1.20 mol) of 1-methylcyclopentene,

200.0 g (6.24 mol) of methanol and

27.1 g of Amberlyst 15. The reaction time was 18.5 h.

A GC sample showed a conversion of 96%.

### Composition of the Product Mixture:

MeOH: 35.5%

3 (1-methylcyclopentene): 2.7%

7 (ether): 60.1%

### Example 12

### Preparation of 1-methoxy-1-methylcyclopentane

### Olefin:MeOH=1/5.1; Short Reaction Time

Procedure analogous to Example 11, using

100.0 g (1.22 mol) of 1-methylcyclopentene,

200.0 g (6.24 mol) of methanol and

27.1 g of Amberlyst 15. The reaction time was 4.6 h.

A GC sample showed a conversion of 96%.

### Composition of the Product Mixture:

MeOH: 33.7%

3 (1-methylcyclopentene): 2.3%

7 (ether): 61.7%

In this example, it is shown that a good conversion/yield is achieved even with reasonable residence times.

### Example 13

### Preparation of 1-methoxy-1-methylcyclopentane

### Without Soxhlet

1-Methylcyclopentene (50.0 g, 0.61 mol), methanol (200.0 g, 6.24 mol) and 25.0 g of Amberlyst 15 were introduced as initial charge in a 3-neck flask under nitrogen. The mixture was heated to 50° C. After 6 h, a GC sample was taken which showed a conversion of ca. 50%. After 24 h, a further GC sample was taken which showed a conversion of ca. 67%. Neither a longer reaction time, nor the addition of 5.0 g of Amberlyst 15 led to a further conversion.

### Example 14

### Preparation of 1-methoxy-1-methylcyclopentane starting from a mixture which comprises components 2, 3, 4 and 5

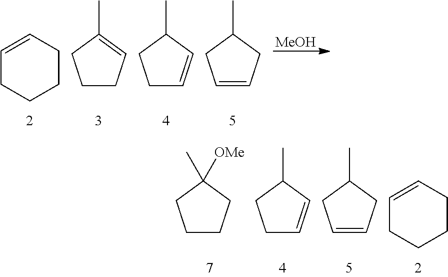

The experiment was carried out analogously to Example 12 with Amberlyst 15 as catalyst. The following starting material mixture was used:

MeOH (31 GC area %), 2 (14 GC area %), 3 (38 GC area %), 4 (10 GC area %), 5 (4 GC area %). After a reaction time of 19 h at 48° C., the following mixture was obtained: MeOH (28 GC area %), 2 (14 GC area %), 3 (15 GC area %), 4 (10 GC area %), 5 (4 GC area %), 7 (25 GC area %). The example shows that only compound 3 reacts to give the product 7. According to GC analysis, compounds 2, 4 and 5 do not react and can be returned to the gas-phase reaction (see Examples 1 to 3).

### Example 15

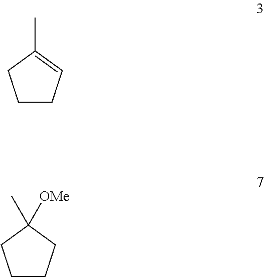

A mixture consisting of compounds 3 and 7 was subjected to fractional distillation at a pressure between 1 bar and 100 mbar.

| | | |
|---|---|---|
| Compounds | | |
| [in GC | Boiling | |
| area %] | point | Pressure |

| Sample | g | 3 | 7 | [° C.] | bar |
|---|---|---|---|---|---|
| Feed | 431.0 | 20.4 | 73.9 | | |
| Fr.1 | 40.7 | 96.0 | 0.9 | 22-35 | 1-0.1 |
| Fr.2 | 34.3 | 79.2 | 19.3 | 35-61 | 1-0.1 |
| Fr.3 | 23.0 | 33.7 | 65.9 | 47-70 | 1-0.1 |
| Fr.4 | 10.4 | 8.5 | 91.2 | 70-71 | 0.1 |
| Fr.5 | 71.6 | 1.2 | 98.6 | 72 | 0.1 |
| Fr.6 | 183.6 | 0.0 | 99.6 | 72 | 0.1 |
| Fr.7 | 27.6 | 0.0 | 99.5 | 72 | 0.1 |
| Bottom | 24.1 | | | | |

The experiment shows the distillative separation of 3 and 7.

1. A process for the preparation of cyclopentane derivatives of the formula I 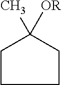
in which R is a C1-C10-alkyl group, which comprises reacting cyclohexanol or cyclohexene or mixtures of both compounds to give 1-methyl-1-cyclopentene (1st stage) and subsequently adding a C1- to C10-alkanol onto the double bond of the 1-methyl-1-cyclopentene (2nd stage).

2. The process according to claim 1, wherein the addition of the C1- to C10 alkanol onto the double bond of the 1-methylcyclopentene takes place by means of a reactive distillation.

3. The process according to claim 1 or 2, wherein the resulting by-products 3-methylcyclopentene and 4-methylcyclopentene from the product mixture of the 1st or 2nd stage are returned to the reaction in the 1st stage.

4. The process according to any one of claims 1 to 3, wherein the process is carried out continuously.

5. The process according to any one of claims 1 to 4, wherein the resulting by-products 3-methylcyclopentene and 4-methylcyclopentene from the product mixture in the 1st stage are returned to the reaction in the 1st stage.

6. The process according to claim 5, wherein 1-methylcyclopentene and optionally water are separated off from the product mixture from the 1st stage, and 3-methylcyclopentene and 4-methylcyclopentene and unreacted cyclohexene are returned to the reaction in the 1st stage.

7. The process according to any one of claim 5 or 6, wherein unreacted 1-methylcyclopentene and alkanol from the product mixture in the 2nd stage are returned to the reaction in the 2nd stage.

8. The process according to any one of claims 1 to 4, wherein
after the 1st stage, no separation off and recycling of 3-methylcyclopentene and 4-methylcyclopentene takes place and
3-methylcyclopentene and 4-methylcyclopentene from the product mixture of the 2nd stage are returned to the reaction in the 1st stage.

9. The process according to claim 8, wherein product mixture from the 2nd stage comprises, besides the desired cyclopentane derivative of the formula I, the following compounds:
unreacted 1-methylcyclopentene
the by-products 3-methylcyclopentene and 4-methylcyclopentene
unreacted cyclohexene
optionally alkanol.

10. The process according to any one of claim 8 or 9, wherein the desired cyclopentane derivative and optionally alkanol are separated off from the product mixture from the 2nd stage, and 3-methylcyclopentene and 4-methylcyclopentene as well as unreacted cyclohexene and 1-methylcyclopentene and optionally alkanol are returned to the reaction in the 1st stage.
